(19) United States
(12) Patent Application Publication
(10) Pub. No.: US 2016/0090333 A1
(43) Pub. Date: Mar. 31, 2016

(54) METHODS AND APPARATUS FOR PRODUCING JET-RANGE HYDROCARBONS

(71) Applicant: UOP LLC, Des Plaines, IL (US)

(72) Inventors: Dana K. Sullivan, Mount Prospect, IL (US); Charles P. Luebke, Mount Prospect, IL (US); Christopher P. Nicholas, Evanston, IL (US); Geoffrey William Fichtl, Chicago, IL (US)

(21) Appl. No.: 14/498,945

(22) Filed: Sep. 26, 2014

(51) Int. Cl. C07C 2/12; C12P 7/02; C07C 7/04; B01J 19/24; C07C 5/03; C07C 1/24

(52) U.S. Cl. CPC C07C 2/12; C07C 5/03; C07C 1/24; C07C 7/04; B01J 19/245; C12P 7/02; C07C 2529/06; C12P 2203/00

(57) ABSTRACT

Methods and apparatus are provided for producing jet-range hydrocarbons from biorenewable sources, such as oligomerizing C₃-C₈ biorenewable olefins, for example, derived from C₃-C₈ alkanol products of fermentation of biomass. Production of jet-range hydrocarbons is increased by employing an additional oligomerization zone for oligomerizing naphtha separated from the effluent of a primary oligomerization zone wherein the C₃-C₈ biorenewable olefins were first subjected to oligomerization.

### FIELD OF THE INVENTION

The present disclosure generally relates to methods and apparatus for producing renewable fuels and chemicals from biorenewable sources, and more particularly relates to methods and apparatus for producing jet-range hydrocarbons from C₃-C₈ biorenewable olefins.

### BACKGROUND

As the worldwide demand for fuel increases, interest in sources other than crude oil from which to produce transportation fuels, including aviation fuels, is ever increasing. For example, due to the growing environmental concerns over fossil fuel extraction and economic concerns over exhausting fossil fuel deposits, there is a demand for using an alternate “green” or “biorenewable” feed source for producing hydrocarbons useful as transportation fuels and in other industries. Suitable biorenewable sources identified to date include vegetable and seed oils, animal fats, biomass fermentation products, and algae byproducts, among others. Various processes are known, and more are being developed, to convert different types of biorenewable feedstock into green fuels that may be used as substitutes for the different types of fuel being produced from crude oil. As used herein, the terms “biorenewable fuel,” “biorenewable jet fuel” and “biorenewable diesel fuel” refer to fuels produced from biorenewable sources, in contrast to those produced from crude oil. The conversion processes often also produce propane and other light hydrocarbons, as well as naphtha or green diesel fuel, in addition to the preferred or target fuel being produced.

Biomass fermentation products typically include lower isoalkanols such as, for example, C₃-C₈ isoalkanols obtained by contacting biomass with biocatalysts that facilitate conversion (by fermentation) of the biomass to isoalkanols of interest. The biomass feedstock for such fermentation processes can be any suitable fermentable feedstock known in the art, such as fermentable sugars derived from agricultural crops including sugarcane, corn, etc. Suitable fermentable biomass feedstock can also be prepared by the hydrolysis of biomass, for example lignocellulosic biomass (e.g. wood, corn stover, switchgrass, herbiage plants, ocean biomass, etc.), to form fermentable sugars.

Jet-range fuels are an important product for the aerospace industry and the military. The specific characteristics of various grades and types of jet-range fuels vary slightly according to the particular application and environment in which they are used. Generally, jet-range fuels are a mixture of primarily C₈-C₁₆ hydrocarbons and typically have a freezing point of about −40 or −47° C. In order to produce jet-range fuels from isoalkanols derived from fermented biomass, in one example known in the art, isobutanol is first dehydrated to form butenes. The butenes are then oligomerized, in the presence of an oligomerization catalyst, in one or more reactors to form heavier olefins, such as C₅-C₂₀, or even higher, olefinic oligomers. Finally, the resulting olefinic oligomers are hydrogenated in a saturation reactor to form the corresponding C₅-C₂₀, or even higher, paraffins in a mixture which can then be subjected to separation to obtain C₉-C₂₀₊ paraffins suitable for use as biorenewable jet fuel.

Since the oligomerization reaction is highly exothermic, the butene fed to the oligomerization reactors may be cooled before entering the oligomerization reactors. Another measure taken to control the temperature increase in the oligomerization reactors is to limit the proportion of olefins contained in the feedstream provided to each reactor to no more than about 15 percent by weight (wt %). This is accomplished, at least in part, by adding non-reactive diluent material to the reactors which also provides a heat sink to control the temperature rise in the reactors.

In the foregoing conversion process, however, it was found that the oligomerization reactors produced a mixed oligomer product containing an undesirable excess of naphtha range olefinic hydrocarbons, i.e., a mixture of primarily C₅-C₁₀ olefins. To address this problem, naphtha range olefinic hydrocarbons were separated from the mixed oligomer product and recycled back to the oligomerization reactors. This resulted in a nearly naphtha-free oligomer product for providing to the saturation reactor, but also resulted in very large volumetric flowrates to each of the oligomerization reactors. This is because the recycled olefins themselves added volume and, furthermore, the additional olefins in the feedstream required additional diluent to be added to the feedstream to keep the olefin content at 15 wt % or less.

Accordingly, it is desirable to provide methods and apparatus for producing jet-range fuels from a biorenewable feedstock in which the naphtha range hydrocarbon content of an oligomerization product is reduced prior to hydrogenation, without increasing the total volume of material provided to the oligomerization stage. Other desirable features and characteristics of the present disclosure will become apparent from the subsequent detailed description and the appended claims, when taken in conjunction with the accompanying drawing and this background.

### SUMMARY OF THE INVENTION

Methods and apparatus for producing jet-range hydrocarbons are disclosed herein. In an exemplary embodiment, a method for producing jet-range hydrocarbons comprises: providing an olefin stream comprising one or more biorenewable C₃-C₈ olefins to a primary oligomerization zone comprising an oligomerization catalyst to produce a primary oligomerized effluent; separating the primary oligomerized effluent to produce a jet-range hydrocarbon stream and a naphtha stream comprising primarily C₅-C₁₀ olefins; and providing the naphtha stream to a naphtha oligomerization zone to produce a secondary oligomerized effluent.

In another exemplary embodiment, a method for producing jet-range hydrocarbons comprises: providing an olefin stream comprising one or more biorenewable C₃-C₈ olefins to a primary oligomerization zone comprising a zeolite catalyst to produce a primary oligomerized effluent; separating the primary oligomerized effluent using a distillation column to produce a jet-range hydrocarbon stream and a naphtha stream comprising primarily C₅-C₁₀ olefins; providing the naphtha stream to a naphtha oligomerization zone to produce a secondary oligomerized effluent; hydrogenating the jet-range hydrocarbon stream in a saturation zone; and providing at least a portion of the secondary oligomerized effluent to: the distillation column, the saturation zone, the naphtha oligomerization zone, or combinations thereof.

In still another exemplary embodiment, an apparatus for producing jet-range hydrocarbons is provided, wherein the apparatus comprises: a primary oligomerization zone comprising an oligomerization catalyst and having the capacity to receive an olefin stream comprising one or more biorenewable C₃-C₈ olefins and produce a primary oligomerized effluent; a distillation column in fluid communication with the primary oligomerization zone and being capable of separating the primary oligomerized effluent and producing a jet-range hydrocarbon stream and a naphtha stream comprising primarily C₅-C₁₀ olefins; and a naphtha oligomerization zone in fluid communication with the distillation column and being capable of receiving the naphtha stream and producing a secondary oligomerized effluent.

### BRIEF DESCRIPTION OF THE DRAWINGS

Exemplary embodiments of the present disclosure will hereinafter be described in conjunction with the following drawing figures, wherein like numerals denote like elements, and like elements in subsequent embodiments are labeled with like numerals increased by 100 with respect to the previous embodiment, and wherein:

FIG. 1 schematically illustrates an exemplary embodiment of an apparatus for producing jet-range hydrocarbons and including a separate naphtha oligomerization zone;

FIG. 2 schematically illustrates another exemplary embodiment of an apparatus for producing jet-range hydrocarbons; and

FIG. 3 schematically illustrates another exemplary embodiment of an apparatus for producing jet-range hydrocarbons.

### DETAILED DESCRIPTION

The following detailed description is merely exemplary in nature and is not intended to limit the invention or the application and uses of the invention. As used herein, the word “exemplary” means “serving as an example, instance, or illustration.” Any embodiment described herein as “exemplary” is not necessarily to be construed as preferred or as advantageous over other embodiments. Furthermore, there is no intention to be bound by any theory presented in the preceding background or the following detailed description.

Disclosed herein are methods and apparatus for producing jet-range hydrocarbons from one or more biorenewable C₃-C₈ olefins via oligomerization. While these methods find greatest utility in converting feedstocks from alkanols, thereby allowing for production of jet fuels from renewable sources, this is not intended to limit the application of the method. As used herein, the terms “jet-range hydrocarbons,” “jet-range paraffins,” “jet-range fuels,” or “jet fuels” refer to a mixture of primarily C₈-C₁₆ hydrocarbons with a freezing point of about −40° C. or about −47° C. As used herein, the phrase “a mixture of primarily . . . ” or “comprising primarily . . . ” a specified range of carbon-numbered hydrocarbons means that the group or category of hydrocarbons being described may also contain very small amounts of hydrocarbons outside the stated carbon number range, without altering the general characteristics (e.g., boiling point) of the group or category being described. For example, the description that jet fuels are a mixture of primarily C₈-C₁₆ hydrocarbons means that jet fuels contain at least 80 weight percent of hydrocarbon molecules each having from about 8 to about 16 carbon atoms with, possibly, very small amounts of hydrocarbon molecules each having less than about 8 carbon atoms, as well as very small amounts of hydrocarbon molecules each having more than 23 carbon atoms, such that the boiling point remains about −40° C. to about −47° C.

There are multiple standards, established by various industries and governments, that are useful for ensuring that particular types of jet fuels have uniform characteristics that fall within expected ranges. For example, one type of jet fuel, known as Aviation Turbine Fuel, Jet A, or Jet A-1 fuel, is composition of hydrocarbons that boil in a range such that the volatility characteristics of the hydrocarbon (or paraffinic form of the hydrocarbon after hydrogenation) substantially conform to the volatility standards of flash point and distillation range set forth in ASTM D7566-11a, “Standard Specification for Aviation Turbine Fuel Containing Synthesized Hydrocarbons,” promulgated by ASTM International, Inc. of West Conshohoken, Pa. Other standards that provide parameters useful for characterizing and defining the jet fuels prepared using the methods and apparatus contemplated and described herein include Jet Propellant (JP)-5 and JP-8, which are set forth in the United States military specifications found at MIL-DTL-83133, as well as in British Defence Standard 91-87.

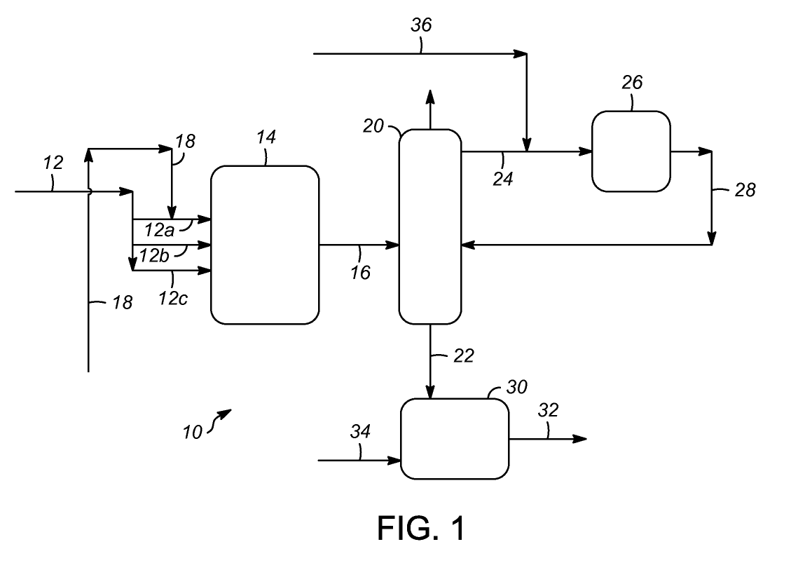

Apparatus for producing jet-range hydrocarbons from an olefin stream comprising one or more renewable C₃-C₈ olefins will now be described with reference to FIGS. 1-3. It is noted that as depicted, process flow lines in FIGS. 1-3 may be referred to interchangeably as, e.g., lines, pipes, conduits, feeds, gases, products, discharges, parts, portions, or streams. The apparatus shown and described are useful for performing the methods for producing jet-range hydrocarbons described in more detail hereinafter.

FIG. 1 provides a schematic illustration of an apparatus 10 for producing jet-range hydrocarbons. More particularly, in the exemplary embodiment shown in FIG. 1, an olefin stream 12 comprising one or more biorenewable C₃-C₈ olefins is provided to a primary oligomerization zone 14 that includes an oligomerization catalyst (not shown per se) capable of catalyzing conversion of the one or more biorenewable C₃-C₈ olefins to higher boiling hydrocarbons including jet-range hydrocarbons. The primary oligomerization zone 14 is capable of receiving the olefin stream 12 and producing a primary oligomerization effluent 16 comprising jet-range hydrocarbons. The primary oligomerization 14 zone may, for example, without limitation, may be operated at a temperature from about 100° C. to about 300° C. and a pressure of from about 689 kiloPascals (“kPa”) (100 pounds per square inch, “psi”) to about 6895 kPa (1000 psig). For example, the operating temperature may be from about 120 to about 280° C., or even from about 160 to about 260° C. The operating pressure may, for example, be from about 1034 kPa (150 psi) to about 5516 kPa (800 psi), or even from about 2068 kPa (300 psi) to about 4964 kPa (720 psi).

A diluent stream 18 is also typically provided to the primary oligomerization zone 14 for controlling the temperature increase caused by the exothermic oligomerization reaction. As will become clearer hereinafter to persons of ordinary skill, the diluent stream 18 may be derived from saturation zone stripper bottoms, distillation column bottoms, or oligomerization zone effluents.

As used herein, the term “stream” can include various hydrocarbon molecules and other substances. Moreover, the term “stream comprising Cₓ hydrocarbons” or “stream comprising Cₓ olefins” can include a stream comprising hydrocarbon or olefin molecules, respectively, with “x” number of carbon atoms, suitably a stream with a majority of hydrocarbons or olefins, respectively, with “x” number of carbon atoms and preferably a stream with at least 75 wt-% hydrocarbons or olefin molecules, respectively, with “x” number of carbon atoms. Moreover, the term “stream comprising Cₓ₊ hydrocarbons” or “stream comprising Cₓ₊ olefins” can include a stream comprising a majority of hydrocarbon or olefin molecules, respectively, with more than or equal to “x” carbon atoms and suitably less than 10 wt-% and preferably less than 1 wt-% hydrocarbon or olefin molecules, respectively, with x−1 or less carbon atoms. Lastly, the term “Cₓ₋ stream” can include a stream comprising a majority of hydrocarbon or olefin molecules, respectively, with less than or equal to “x” carbon atoms and suitably less than 10 wt-% and preferably less than 1 wt-% hydrocarbon or olefin molecules, respectively, with x+1 or greater carbon atoms.

In some embodiments of the method contemplated and described herein, biorenewable C₃-C₈ isoalkanols derived from fermentation biorenewable sources may be dehydrated to form the olefin stream comprising one or more biorenewable C₃-C₈ olefins that is provided to the primary oligomerization zone 14. More particularly, for example, isopropanol (i.e., C₃ isoalkanol) formed by fermentation of biomass may be converted by dehydration to propene (i.e., the corresponding C₃ olefin) for providing to the primary oligomerization zone 14. Similarly, isobutanol (i.e., C₄ isoalkanol) formed by fermentation of biomass or by condensation reactions of synthesis gas, may be dehydrated to form isobutene (i.e., the corresponding C₄ olefin). Furthermore, in embodiments where both isopropanol and isobutanol are both available, they may both be subjected to dehydration to form a mixture of C₃-C₄ olefins for the olefin stream to be provided to the primary oligomerization zone 14. The feedstock used in the fermentation process to produce one or more biorenewable C₃-C₈ isoalkanols can be any suitable fermentable feedstock known in the art, including fermentable sugars derived from agricultural crops including sugarcane, corn, or from lignocellulosic biomass (e.g. wood, corn stover, switchgrass, herbiage plants, ocean biomass, etc.).

In another example, biorenewable alkanols, such as isobutanols, can be prepared photosynthetically, for example using cyanobacteria or algae engineered to produce isobutanol and/or other alkanols. When produced photosynthetically, the feedstock for producing the resulting renewable alkanols is light, water, and CO₂, which is provided to the photosynthetic organism (e.g., cyanobacteria or algae). Additionally, other known methods, whether biorenewable or otherwise, for producing C₃-C₈ isoalkanols, such as isobutanol, are suitable for supplying the olefin stream; the methods described herein are not intended to be limited by the use of any particular biorenewable feed source.

As used herein, the term “zone” can refer to an area including one or more equipment items and/or one or more sub-zones. Equipment items can include one or more reactors or reactor vessels, heaters, exchangers, pipes, pumps, compressors, controllers and columns Additionally, an equipment item, such as a reactor, dryer, or vessel, can further include one or more zones or sub-zones.

The oligomerization catalyst (not shown per se in FIG. 1) contained in the primary oligomerization zone 14 is not particularly limited and must be capable of catalyzing conversion of the one or more biorenewable C₃-C₈ olefins in the olefin stream to olefinic oligomers comprising heavier boiling C₅₊ hydrocarbons, including jet-range hydrocarbons.

The oligomerization catalyst may be any such catalyst known now or in the future. Conventional oligomerization catalysts will generally convert an olefin to a mixture of dimers, trimers, tetramers, and sometimes pentamers, of the olefin. For example, where the C₃-C₈ olefin is isobutanol, a C₄ olefin, the products of oligomerization in the presence of a conventional oligomerization catalyst include C₈, C₁₂, C₁₆, and sometimes C₂₀ olefins, together in a mixture. Conventional oligomerization catalyst include, without limitation, solid phosphoric acid (“SPA”) and certain ion exchange resins such as Amberlyst-36 (commercially available from The Dow Chemical Company of Midland, Mich., U.S.A.). The olefinic oligomer mixture produced using conventional oligomerization catalysts may be further subjected to a separation process to produce a mixture of jet-range hydrocarbons suitable for use as jet fuels. These jet fuels often have a boiling point distribution that has well-defined boiling point steps corresponding to only a few isomers of the corresponding trimer, tetramer, and pentamer paraffins of the starting olefin, which is different from petroleum-derived jet fuels.

Alternative oligomerization catalysts comprising zeolite materials, on the other hand, catalyze oligomerization conversion of C₃-C₈ olefins to dimers, trimers, tetramers, and sometimes pentamers of the C₃-C₈ olefins, but also catalyze backcracking conversion of the resulting heavier olefinic oligomers back into lighter and more random and varied sizes of olefins including C₅-C₂₀₊ hydrocarbons. In other words, under appropriate conditions, zeolitic catalysts such as, without limitation, MTT, TON, MFI, and MTW, yield C₅₊ hydrocarbons, including jet-range hydrocarbons, with an increased distribution and variety of carbon numbers than those made using conventional non-zeolitic catalysts. This means that jet-range fuel produced from biorenewable olefins via oligomerization in the presence of zeolite catalysts has a boiling range and compositional profile that is more similar to jet-range fuels produced from petroleum refining processes.

Suitable zeolite catalysts may comprise between 5 and 95 wt % of zeolite material. Suitable zeolite materials include zeolites having a structure from one of the following classes: MFI, MEL, ITH, IMF, TUN, FER, BEA, FAU, BPH, MEI, MSE, MWW, UZM-8, MOR, OFF, MTW, TON, MTT, AFO, ATO, and AEL. 3-letter codes indicating a zeotype are as defined by the Structure Commission of the International Zeolite Association and are maintained at http://www.iza-structure.org/databases/. UZM-8 is as described in U.S. Pat. No. 6,756,030. In a preferred aspect, the zeolite catalyst may comprise a zeolite with a framework having a ten-ring pore structure. Examples of suitable zeolites having a ten-ring pore structure include TON, MTT, MFI, MEL, AFO, AEL, EUO and FER. In a further preferred aspect, the oligomerization catalyst comprising a zeolite having a ten-ring pore structure may comprise a uni-dimensional pore structure. A uni-dimensional pore structure indicates zeolite materials containing non-intersecting pores that are substantially parallel to one of the axes of the crystal. The pores preferably extend through the zeolite crystal. Suitable examples of zeolite materials having a ten-ring uni-dimensional pore structure may include MTT. In a further aspect, the oligomerization catalyst comprises an MTT zeolite.

The zeolite catalyst may be formed by combining the zeolite material with a binder, and then forming the catalyst into pellets. The pellets may optionally be treated with a phosphorus reagent to create a zeolite having a phosphorous component between 0.5 and 15 wt % of the treated catalyst. The binder is used to confer hardness and strength on the catalyst. Binders include alumina, aluminum phosphate, silica, silica-alumina, zirconia, titania and combinations of these metal oxides, and other refractory oxides, and clays such as montmorillonite, kaolin, palygorskite, smectite and attapulgite. A preferred binder is an aluminum-based binder, such as alumina, aluminum phosphate, silica-alumina and clays.

One of the components of the zeolite catalyst binder utilized herein is alumina. The alumina source may be any of the various hydrous aluminum oxides or alumina gels such as alpha-alumina monohydrate of the boehmite or pseudo-boehmite structure, alpha-alumina trihydrate of the gibbsite structure, beta-alumina trihydrate of the bayerite structure, and the like. A suitable alumina is available from UOP LLC under the trademark Versal. A preferred alumina is available from Sasol North America Alumina Product Group under the trademark Catapal. This material is an extremely high purity alpha-alumina monohydrate (pseudo-boehmite) which after calcination at a high temperature has been shown to yield a high purity gamma-alumina.

A suitable zeolite catalyst may be, for example, prepared by mixing proportionate volumes of zeolite and alumina to achieve the desired zeolite-to-alumina ratio. In an embodiment, the MTT content may about 5 to 85, for example about 20 to 82 wt % MTT zeolite, and the balance alumina powder will provide a suitably supported catalyst. A silica support is also contemplated.

Monoprotic acid such as nitric acid or formic acid may be added to the mixture in aqueous solution to peptize the alumina in the binder. Additional water may be added to the mixture to provide sufficient wetness to constitute a dough with sufficient consistency to be extruded or spray dried. Extrusion aids such as cellulose ether powders can also be added. A preferred extrusion aid is available from The Dow Chemical Company under the trademark Methocel.

The paste or dough may be prepared in the form of shaped particulates, with the preferred method being to extrude the dough through a die having openings therein of desired size and shape, after which the extruded matter is broken into extrudates of desired length and dried. A further step of calcination may be employed to give added strength to the extrudate. Generally, calcination is conducted in a stream of air at a temperature from about 260° C. (500° F.) to about 815° C. (1500° F.). The MTT catalyst is not selectivated to neutralize acid sites such as with an amine.

The extruded particles may have any suitable cross-sectional shape, i.e., symmetrical or asymmetrical, but most often have a symmetrical cross-sectional shape, preferably a spherical, cylindrical or polylobal shape. The cross-sectional diameter of the particles may be as small as 40 μm; however, it is usually about 0.635 mm (0.25 inch) to about 12.7 mm (0.5 inch), preferably about 0.79 mm ( 1/32 inch) to about 6.35 mm (0.25 inch), and most preferably about 0.06 mm ( 1/24 inch) to about 4.23 mm (⅙ inch).

With reference back to FIG. 1, in some embodiments of the apparatus 10, the primary oligomerization zone 14 includes two or more, for example three, oligomerization zones (not shown) contained within separate oligomerization reactors (not shown individually) which are typically connected to one another in series. Each of the two or more oligomerization zones contain an oligomerization catalyst, as described above, for conversion of one or more biorenewable C₃-C₈ olefins in the olefin stream to heavier C₅-C₂₀₊ olefinic oligomers including jet-range hydrocarbons. While the oligomerization catalyst in each of the two or more oligomerization zones are generally the same as one another, they may be different in composition, type, quantity, activity, etc. Furthermore, in such embodiments, the olefin stream 12 is split into two or more olefin feedstreams, for example three olefin feedstreams 12a, 12b, 12c, as shown in FIG. 1. Process conditions for the primary oligomerization zone 14 are optimized to produce a higher percentage of jet range hydrocarbon olefins which, when hydrogenated in subsequent steps as will be described below, result in a desirable jet-range hydrocarbon product. The primary oligomerization 14 zone may, for example, without limitation, may be operated at a temperature from about 100° C. to about 300° C. and a pressure of from about 689 kiloPascals (“kPa”) (100 pounds per square inch, “psi”) to about 6895 kPa (1000 psig). For example, the operating temperature may be from about 120 to about 280° C., or even from about 160 to about 260° C. The operating pressure may, for example, be from about 1034 kPa (150 psi) to about 5516 kPa (800 psi), or even from about 2068 kPa (300 psi) to about 4964 kPa (720 psi).

With reference still to FIG. 1, the primary oligomerized effluent 16 is provided to a separation zone, such as a distillation column 20. The distillation column 20 is in fluid communication with the primary oligomerization zone 14 and is capable of separating the primary oligomerized effluent 16 and producing at least two streams: a jet-range hydrocarbon stream 22 and a naphtha stream 24 comprising primarily C₅-C₁₀ olefins, such as C₈ olefins. As used herein, the term “column” means a distillation column or columns for separating one or more components of different volatilities. Unless otherwise indicated, a column is assumed to include a condenser on an overhead of the column to condense and reflux a portion of an overhead stream back to the top of the column and a reboiler at a bottom of the column to vaporize and send a portion of a bottom stream back to the bottom of the column Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column. Additionally, the term “vapor,” as used herein, means a gas or a dispersion that may include or consist of one or more hydrocarbons. As used herein, the term “overhead stream” can mean a stream withdrawn at or near a top of a vessel, such as a column. As used herein, the term “bottom stream” can mean a stream withdrawn at or near a bottom of a vessel, such as a column. A vent stream 26 comprising lighter hydrocarbons than naphtha range may also be produced by the distillation column 20 and often exits the column 20 as a vapor.

The apparatus 10 for producing jet-range hydrocarbons further comprises a naphtha oligomerization zone 26 which is separate and in addition to the primary oligomerization zone 14 and which also comprises an oligomerization catalyst. The naphtha oligomerization zone 26 is in fluid communication with the distillation column 20 and is capable of receiving the naphtha stream 24 from the distillation column 20. Furthermore, the naphtha oligomerization zone 26 is capable of converting the naphtha range hydrocarbons in the naphtha stream 24 to heavier hydrocarbons, including jet-range hydrocarbons, and producing a secondary oligomerized effluent 28 comprising jet-range hydrocarbons. While the oligomerization catalyst in the naphtha oligomerization zone is generally the same as the oligomerization catalyst in the primary oligomerization zone, these oligomerization catalysts may be different from one another in composition, type, quantity, activity, etc. A diluent 36 is also typically provided to the naphtha oligomerization zone 26 to control temperature increase therein during oligomerization of the naphtha range olefins 24. Again, the diluent stream 36 may be derived from saturation zone stripper bottoms, distillation column bottoms, or oligomerization zone effluents.

As shown in FIG. 1, in some embodiments of the apparatus 10, a portion of the secondary oligomerized effluent 28 is recycled to the distillation column 20. It is noted that the entire secondary oligomerized effluent 28, may be recycled to the distillation column 20, or only a portion, large or small, may be recycled. In such embodiments, the distillation column 20 is in fluid communication with the naphtha oligomerization zone 26 to receive the recycled secondary oligomerized effluent 28. In such a flow scheme, the opportunity is provided for the distillation column 24 to separate additional naphtha range hydrocarbons that may remain in the secondary oligomerized effluent 28, as well as to separate the jet-range hydrocarbons formed in the naphtha oligomerization zone 26 and add them to the jet-range hydrocarbon stream 22.

The apparatus 10 for producing jet-range hydrocarbons, as shown in FIG. 1, may further comprise a saturation zone 30 in fluid communication with the distillation column 20 and the naphtha oligomerization zone 26. The saturation zone 30 is capable of receiving the jet-range hydrocarbon stream 22 from the distillation zone 20 and as well as at least a portion of the secondary oligomerized effluent 28, which also comprises jet-range hydrocarbons. The jet-range hydrocarbons entering the saturation zone 30 are olefinic hydrocarbons and are converted in the saturation zone 30 by hydrogenation to jet-range paraffin hydrocarbons 32 suitable for use as biorenewable jet-range fuels.

Hydrogenation is typically performed in the saturation zone 30 using a conventional hydrogenation or hydrotreating catalyst (not shown), which may include metallic catalysts containing, e.g., palladium, rhodium, nickel, ruthenium, platinum, rhenium, cobalt, molybdenum, or combinations thereof, and the supported versions thereof. Catalyst supports can be any solid, inert substance including, but not limited to, oxides such as silica, alumina, titania, calcium carbonate, barium sulfate, and carbons. The catalyst support can be in the form of powder, granules, pellets, or the like. A stream of hydrogen-containing gas 34 may be provided to the saturation zone 30 as the source of for the hydrogenation reaction. In an exemplary embodiment, the hydrogenation catalyst is a platinum-on-alumina catalyst, for example 0.7 wt. % platinum-on-alumina catalyst. Using this catalyst, hydrogenation suitably occurs in the saturation zone 30 at a temperature of about 150° C. and at a pressure of about 1000 psig. According to these process conditions, in the saturation zone 30, the olefins are converted into paraffin products having the same carbon number distribution as the olefins, thereby forming jet-range paraffins 32 suitable for use as jet-range fuels.

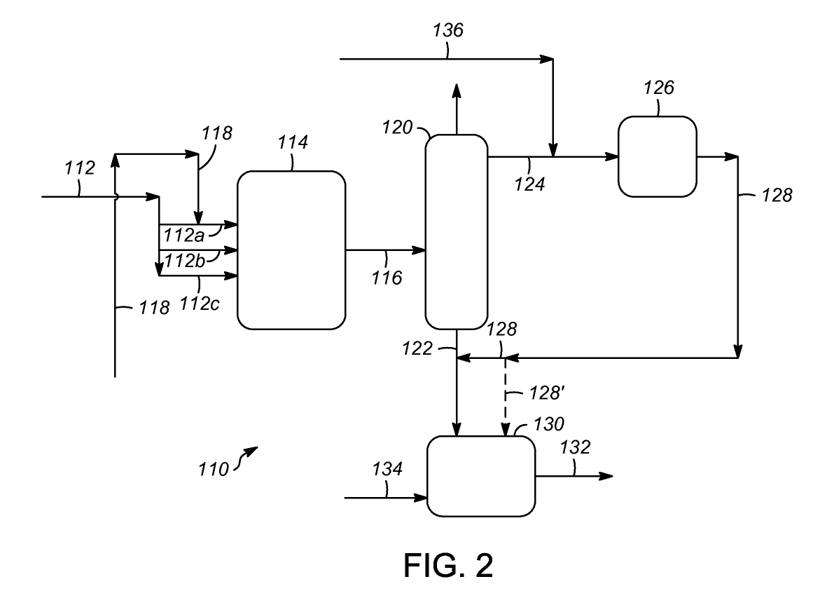

In another exemplary embodiment of the apparatus 110 for producing jet-range hydrocarbons, as shown in FIG. 2, at least a portion of the secondary oligomerized effluent 128 may be provided directly to the saturation zone 130 (see dashed arrow 128′) for conversion to additional jet-range paraffin hydrocarbons 132. Alternatively, and preferably, at least a portion of the secondary oligomerized effluent 128 is combined (see solid arrow 128) with jet-range hydrocarbon stream 122 from the distillation column 120, and then provided to the saturation zone 130 for conversion to jet-range paraffins 132 by hydrogenation.

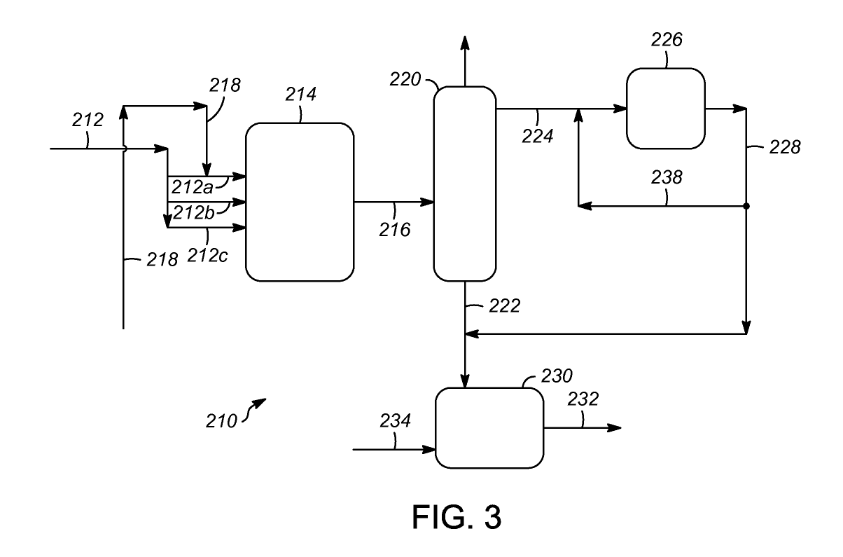

In still another exemplary embodiment of the apparatus 210 for producing jet-range hydrocarbons, as shown in FIG. 3, instead of the diluent stream derived from other process streams, at least a portion 238 of the secondary oligomerized effluent 228 may be recycled directly back to the naphtha oligomerization zone 226 to provide diluent for temperature control. This also provides an opportunity for unconverted naphtha range hydrocarbons in the recycled portion 238 of the secondary oligomerized effluent 228 to be converted to heavier hydrocarbons in the naphtha oligomerization zone 226.

Methods for producing jet-range hydrocarbons from biorenewable feedstock are also provided and will now be described in detail. In an exemplary embodiment, the method for producing jet-range hydrocarbons comprises providing an olefin stream comprising one or more biorenewable C₃-C₈ olefins to a primary oligomerization zone comprising an oligomerization catalyst to produce a primary oligomerized effluent; separating the primary oligomerized effluent to produce a jet-range hydrocarbon stream and a naphtha stream comprising primarily C₅-C₁₀ olefins; and providing the naphtha stream to a naphtha oligomerization zone to produce a secondary oligomerized effluent. Suitable olefin streams and suitable biorenewable C₃-C₈ olefins contained therein are as described above in connection with the apparatus for producing jet-range hydrocarbons.

More particularly, the primary oligomerization zone includes an oligomerization catalyst (not shown per se) capable of catalyzing conversion of the biorenewable C₄ olefins to higher boiling hydrocarbons including jet-range-hydrocarbons. Suitable oligomerization catalysts have already been described above and include conventional oligomerization catalysts as well as zeolite catalysts. The primary oligomerization zone may include two or more, oligomerization zones, contained within separate oligomerization reactors arranged in series with one another. Each of the two or more oligomerization zones should contain an oligomerization catalyst, which may be the same or different from one another, as described above, for conversion of the one or more biorenewable C₃-C₈ olefins in the olefin stream to jet-range hydrocarbons. To accommodate multiple oligomerization zones, the olefin stream 12 may be split into multiple olefin streams, i.e., one for each oligomerization zone (e.g., see FIG. 1, three olefin streams 12a, 12b, 12c). Suitable operating conditions for the primary oligomerization zone are as stated above for the primary oligomerization zone 14 of the system of FIG. 1. Regardless of the particular configuration of the primary oligomerization zone, a primary oligomerization effluent comprising jet-range hydrocarbons is produced and exits the primary oligomerization zone.

A diluent stream is also typically provided to the primary oligomerization zone 14 for controlling the temperature increase caused by the exothermic oligomerization reaction. As will be understood by persons of ordinary skill in the relevant art based on the foregoing descriptions of the apparatus for producing jet-range hydrocarbons, the diluent stream 18 may be derived from saturation zone stripper bottoms, distillation column bottoms, or oligomerization zone effluents.

The primary oligomerized effluent from the primary oligomerization zone is separated to produce a jet-range hydrocarbon stream and a naphtha stream comprising C₈ olefins. The method of separating the primary oligomerized effluent is not particularly limited and may be any method known now or in the future to persons of ordinary skill in the relevant art. A suitable separation method produces a stream comprising jet-range hydrocarbons suitable for use as jet-range fuel and another separate stream comprising naphtha range hydrocarbons, including but not limited to C₅-C₁₀ hydrocarbons. For example, without limitation, as described above in connection with the apparatus shown in FIG. 1, a distillation column may be used to perform the separation of the primary oligomerized effluent.

The method contemplated and described herein further comprises providing the naphtha stream to a naphtha oligomerization zone, which is different and separate from the primary oligomerization zone, to produce a secondary oligomerized effluent comprising higher boiling olefinic oligomers, including jet-range hydrocarbons. Thus, rather than simply recycling the naphtha stream to the primary oligomerization zone for conversion of the naphtha range olefins to higher boiling olefinic oligomers, the naphtha stream is provided to a separate dedicated naphtha oligomerization zone for such conversion. This arrangement produces more of the desired higher boiling olefinic oligomers from the naphtha range hydrocarbons in the naphtha stream, but avoids increasing volumetric flow to the primary oligomerization zone otherwise caused by simple recycle of the entire naphtha stream to the primary oligomerization zone. Using a separate naphtha oligomerization zone according to the aforesaid method also avoids adding more olefins to feedstreams that must be maintained below about 15-25 wt % olefins as they enter the primary oligomerization zone to control temperature in the primary oligomerization zone. All or a portion of the secondary oligomerized effluent from the naphtha oligomerization zone may be recycled to the separation stage (e.g., the distillation column used to perform the separation), or directly back to the naphtha oligomerization zone, for more complete conversion of the naphtha to higher boiling hydrocarbons.

Operating conditions used in the naphtha oligomerization zone may be the same or different as those utilized in the primary oligomerization zone. The naphtha oligomerization zone may, for example, without limitation, may be operated at a temperature from about 100° C. to about 300° C. and a pressure of from about 689 kiloPascals (“kPa”) (100 pounds per square inch, “psi”) to about 6895 kPa (1000 psig). For example, the operating temperature may be from about 120 to about 280° C., or even from about 160 to about 260° C. The operating pressure may, for example, be from about 1034 kPa (150 psi) to about 5516 kPa (800 psi), or even from about 2068 kPa (300 psi) to about 4964 kPa (720 psi). In some embodiments, the pressure in the naphtha oligomerization zone is lower than that in the primary oligomerization zone. In some embodiments, the temperature in the naphtha reaction zone is lower than the temperature in the primary oligomerization zone.

In another exemplary embodiment, the method contemplated and described herein may further include hydrogenating the jet-range hydrocarbon stream in a saturation zone. More particularly, when the primary oligomerized effluent from the primary oligomerization zone is separated, the resulting jet-range hydrocarbon stream is subjected to hydrogenation to saturate the jet-range hydrocarbons therein to form jet-range paraffins useful as jet-range fuels. The saturation zone typically comprises a conventional hydrogenation catalyst to catalyze the conversion of the olefins to paraffins. In such embodiments that include hydrogenating the jet-range hydrocarbon stream in a saturation zone, the method may further comprise providing at least a portion of the secondary oligomerized effluent to the saturation zone. For example, without limitation, at least a portion of the secondary oligomerized effluent may be combined with the jet-range hydrocarbon stream produced by the separation step, prior to providing the jet-range hydrocarbon stream to the saturation zone. Additionally, in embodiments where at least a portion of the secondary oligomerized effluent is provided to the saturation zone, the method may further comprise providing at least another portion of the secondary oligomerized effluent may be recycled to the naphtha oligomerization zone.

### Examples

The composition of the mixed product stream resulting from practicing the methods and apparatus in accordance with those contemplated and described herein, have been predicted by conventional calculation of mass balances for process streams according to the apparatus shown in FIGS. 1-3 using Microsoft Excel 2007, which is commercially available from Microsoft Corporation of Redmond, Wash., USA, as well as many software retailers worldwide.

The operating conditions and results, as predicted by the mass balance calculations modeling software, for each embodiment of an apparatus in accordance with the various methods for producing jet-range hydrocarbons are provided below in Table 1. The general apparatus is the same for each embodiment and includes a primary oligomerization zone having three oligomerization reactors arranged in series with one another and each containing a zeolite type of oligomerization catalyst. According to a general embodiment of the method described hereinabove, the effluent of the primary oligomerization zone is separated in a distillation column to form a jet-range hydrocarbon stream and a naphtha stream. The apparatus used for the embodiments of the second and third columns (from the left) in Table 1 are comparative and do not include a separate naphtha oligomerization zone. The right-most three columns in Table 1 use an apparatus that does include a separate naphtha oligomerization zone (26, 126, 226) that receives and oligomerizes a naphtha stream (24, 124, 224, respectively) from the distillation column (20, 120, 220, respectively) from which to produce a secondary oligomerized effluent (28, 128, 228, respectively) which is recycled in various ways according to the various embodiments of the method for producing jet-range hydrocarbons. Finally, the jet-range hydrocarbons (22, 122, 222) from the distillation column are generally olefinic and are sent to a saturation zone (30, 130, 230) for conversion to paraffinic jet-range hydrocarbons by hydrogenation.

More particularly, the second from left column of Table 1 demonstrates providing an olefin stream to an oligomerization zone, the effluent of which is separated to form a jet-range hydrocarbon stream and a naphtha stream, followed by recycling the naphtha stream to the oligomerization zone. The third from left column demonstrates the pre-existing operation scheme where an olefin stream is provided to the oligomerization zone, the effluent of which is separated to form a jet-range hydrocarbon stream and a naphtha stream, but the naphtha stream is withdrawn from the apparatus without any recycle.

The fourth from left column in Table 1 demonstrates the results obtained from operating apparatus that includes a separate naphtha oligomerization zone as shown in FIG. 2. In particular, the secondary oligomerized effluent (128) from the separate naphtha oligomerization zone (126) is provided to the saturation zone (130), via combination first with the jet-range hydrocarbon stream (122) from the distillation column (120).

The fifth from left column in Table 1 demonstrates operation of the same apparatus as the fourth from left column, but as shown in FIG. 3. More particularly, a portion (238) of the secondary oligomerized effluent (228) is recycled to the naphtha oligomerization zone (226) as diluent, and the remainder of the secondary oligomerized effluent (228) is provided to the saturation zone (230), via combination first with the jet-range hydrocarbon stream (222) the distillation column (220).

The sixth from left column in Table 1 demonstrates operation of the same apparatus as the third and fourth columns from left, but as shown in FIG. 1. More particularly, the secondary oligomerized effluent (28) is simply recycled to the distillation column (20), while the distillation column (20) supplied with separate diluent (36).

TABLE 1

| | | | | | FIG. 1 + |
|---|---|---|---|---|---|
| | | | | | Naphtha |
| | | | FIG. 2 + | | Rx |
| | | | Naphtha Rx | FIG. 3 + | w/Separate |
| | Recycle | Once- | with Separate | Naphtha Rx | Diluent, |
| | Naphtha | Through | Diluent, | with Naphtha | Effluent |
| NET YIELDS, WT % | to Main | (no | Effluent to | Rx Effluent as | Recycled to |
| by Hydrocarbon Fraction, | Olig | Naphtha | Saturation Unit | Diluent | Splitter |
| Based on FF OLEFS | Reactors | Recycle) | [(122) + (128)] | [(222) + (228)] | [(22)] |
| Lights, C₁-C₄ | 4.15 | 5.62 | 3.67 | 3.70 | 3.70 |
| Naphtha, C₅-C₁₀ | 0.25 | 20.76 | 3.36 | 0.44 | 0.25 |
| Jet, C₈-C₁₆ | 93.2 | 65.95 | 84.63 | 87.45 | 87.63 |
| Diesel + Heavies, C₁₆_{₊} | 2.4 | 7.67 | 8.34 | 8.41 | 8.42 |
| Total | 100.00 | 100.00 | 100.00 | 100.00 | 100.00 |
| (jet + diesel + heavies) | 95.60 | 73.63 | 92.97 | 95.86 | 96.05 |
| Flow to Main Rx 1 if FF = 100, wt/hr | 317 | 181 | 181 | 181 | 181.00 |
| Flow to Naphtha Rx [(24) + (36)] if FF = 100, wt/hr | | | 133.35 | 177.22 | 157.38 |
| Flow to Splitter [(16) + (28)] if FF = 100, wt/hr | 443 | — | 254 | 254 | 412 |
| Total Sat Diluent Used, wt/hr | 268 | 154 | 268 | 154 | 288 |
| Olef in Total Feed to Main Rx 1 if FF = 100, wt/hr | 48.9 | 27.2 | 27.2 | 27.2 | 27.2 |
| Olef in Total Feed to Main Rx 2 if FF = 100, wt/hr | 57.6 | 32.1 | 32.1 | 32.1 | 32.1 |
| Olef in Total Feed to Main Rx 3 if FF = 100, wt/hr | 68.1 | 37.9 | 37.9 | 37.9 | 37.9 |

KEY: Reference numbers (##) relate to FIG. 1, unless otherwise indicated
Rx = reactor
FF = fresh olefin feed
Olef = one or more C₃-C₈ olefins
Main Rx = Primary Oligomerization Zone (14) includes three reactors: Rx 1, Rx 2 and Rx 3
Naphtha Rx = secondary oligomerization zone (26)
Splitter = separation zone/distillation column (20) that receives primary oligomerization effluent stream

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method for producing jet-range hydrocarbons comprising:
providing an olefin stream comprising one or more biorenewable C₃-C₈ olefins to a primary oligomerization zone comprising an oligomerization catalyst to produce a primary oligomerized effluent;
separating the primary oligomerized effluent to produce a jet-range hydrocarbon stream and a naphtha stream comprising primarily C₅-C₁₀ olefins; and
providing the naphtha stream to a naphtha oligomerization zone to produce a secondary oligomerized effluent, wherein the naphtha oligomerization zone comprises an oligomerization catalyst which is the same or different from the oligomerization catalyst of the primary oligomerization zone.

2. The method of claim 1, further comprising recycling at least a portion of the secondary oligomerized effluent to the naphtha oligomerization zone.

3. The method of claim 1, wherein separating the primary oligomerization effluent is performed with a distillation column, said method further comprising recycling at least a portion of the secondary oligomerized effluent to the distillation column.

4. The method of claim 1, further comprising hydrogenating the jet-range hydrocarbon stream in a saturation zone.

5. The method of claim 4, further comprising providing a portion of the secondary oligomerized effluent to the saturation zone.

6. The method of claim 4, further comprising recycling another portion of the secondary oligomerized effluent to the naphtha oligomerization zone.

7. The method of claim 5, wherein providing the portion of the secondary oligomerized effluent to the saturation zone comprises combining the portion of the secondary oligomerized effluent with the jet-range hydrocarbon stream prior to providing the jet-range hydrocarbon stream to the saturation zone.

8. The method of claim 7, further comprising recycling another portion of the secondary oligomerized effluent to the naphtha oligomerization zone.

9. The method of claim 1, further comprising dehydrating one or more biorenewable C₃-C₈ alkanols to form the olefin stream comprising one or more biorenewable C₃-C₈ olefins.

10. The method of claim 9, further comprising deriving the one or more biorenewable C₃-C₈ alkanols from fermentation of biomass.

11. The method of claim 1, wherein providing the olefin stream to the primary oligomerization zone comprises providing the olefin stream to the primary oligomerization zone, wherein the oligomerization catalyst of both the primary and naphtha oligomerization zones is a zeolite catalyst comprising a TON, MTW, MTT or MFI type zeolite.

12. The method of claim 1, wherein the oligomerization zone is operated at a temperature from about 100° C. to about 230° C. and a pressure of from about 2068 kPa (300 psig) to about 6895 kPa (1000 psig).

13. The method of claim 1, further comprising providing a diluent stream to the primary oligomerization zone for controlling the temperature increase caused by the exothermic oligomerization reaction.

14. A method for producing jet-range hydrocarbons comprising:
providing an olefin stream comprising one or more biorenewable C₃-C₈ olefins to a primary oligomerization zone comprising an oligomerization catalyst to produce a primary oligomerized effluent;
separating the primary oligomerized effluent using a distillation column to produce a jet-range hydrocarbon stream and a naphtha stream comprising primarily C₅-C₁₀ olefins;
providing the naphtha stream to a naphtha oligomerization zone to produce a secondary oligomerized effluent, wherein the naphtha oligomerization zone comprises an oligomerization catalyst which is the same or different from the oligomerization catalyst of the primary oligomerization zone;
hydrogenating the jet-range hydrocarbon stream in a saturation zone; and
providing at least a portion of the secondary oligomerized effluent to: the distillation column, the saturation zone, the naphtha oligomerization zone, or combinations thereof.

15. An apparatus for producing jet-range hydrocarbons, wherein the apparatus comprises:
a primary oligomerization zone comprising an oligomerization catalyst and having the capacity to receive an olefin stream comprising one or more biorenewable primarily C₃-C₈ olefins and produce a primary oligomerized effluent;
a distillation column in fluid communication with the primary oligomerization zone and being capable of separating the primary oligomerized effluent and producing a jet-range hydrocarbon stream and a naphtha stream comprising C₅-C₁₀ olefins; and
a naphtha oligomerization zone in fluid communication with the distillation column and being capable of receiving the naphtha stream and producing a secondary oligomerized effluent, wherein the naphtha oligomerization zone comprises an oligomerization catalyst which is the same or different from the oligomerization catalyst of the primary oligomerization zone.

16. The apparatus of claim 15, wherein the distillation column is also in fluid communication with the naphtha oligomerization zone for receiving recycled secondary oligomerized effluent from the naphtha oligomerization zone.

17. The apparatus of claim 15, wherein the oligomerization catalyst in both the primary and naphtha oligomerization zones is a zeolite catalyst comprising a TON, MTW, MTT or MFI type zeolite.

18. The apparatus of claim 15, wherein the oligomerization zone comprises two or more oligomerization reactors arranged in series and in fluid communication with one another, and wherein each of the two or more oligomerization reactors comprise an oligomerization catalyst.

19. The apparatus of claim 15, further comprising a saturation zone in fluid communication with both the primary oligomerization zone and the naphtha oligomerization zone and being capable of receiving the primary and secondary oligomerized effluents and producing biorenewable jet range hydrocarbons.

20. The apparatus of claim 19, wherein the naphtha oligomerization zone comprises a recycle conduit for recycling at least a portion of the secondary oligomerized effluent from the naphtha oligomerization zone back to the naphtha oligomerization zone.
